(19) United States
(12) Patent Application Publication
(10) Pub. No.: US 2011/0105981 A1
(43) Pub. Date: May 5, 2011

(54) DIALYSIS DEVICE

(75) Inventors: Steffan Wagner, Messstetten (DE); Ralf Flieg, Rangendingen (DE)

(73) Assignee: Gambro Lundia AB, Lund (SE)

(21) Appl. No.: 12/911,312

(22) Filed: Oct. 25, 2010

(30) Foreign Application Priority Data
Nov. 3, 2009 (DE) 10 2009 051 805.3

(51) Int. Cl. A61M 1/14; A61M 1/28

(52) U.S. Cl. 604/5.04; 604/29

(57) ABSTRACT

The present invention relates to dialysis devices comprising peristaltic pumps of linear build with piezoelectric actuators.

### FIELD OF THE INVENTION

The present invention relates to dialysis devices comprising peristaltic pumps of linear build with piezoelectric actuators.

### BACKGROUND OF THE INVENTION

In present-day dialysis systems, liquid transport is normally carried out by radial peristaltic pumps which are employed to pump blood, dialysis liquid or liquids or concentrates used for its preparation, and filtrate.

Disadvantages of the radial peristaltic pumps used up to now are their installation size and weight, which contribute to the considerable space requirement and weight of conventional dialysis systems, causing these systems to often be very bulky and heavy and cumbersome to transport. In addition to that, this type of pumps produces an unpleasant noise level during operation and shows high power consumption and heat generation associated with it.

It would therefore be desirable, especially also in view of the development of systems for home dialysis or the development of portable artificial kidneys, to equip devices with alternative pump systems which do not show said disadvantages.

### SUMMARY OF THE INVENTION

It has now been found that peristaltic pumps of linear build with piezoelectric actuators can advantageously replace the conventional radial peristaltic pumps in dialysis systems.

The present invention provides dialysis systems comprising peristaltic pumps of linear build with piezoelectric actuators. The dialysis systems of the invention are distinguished from state-of-the-art systems by smaller installation size and lower weight, they produce virtually no noise and show low power consumption and thus also low heat generation.

Another object of the invention is the use of peristaltic pumps of linear build with piezoelectric actuators in dialysis systems.

### SHORT DESCRIPTION OF THE DRAWINGS

FIG. 1 shows a schematic of an embodiment of a dialysis system of the present invention, suitable for use in intense care;

FIGS. 2a and 2b show schematics of other embodiments of a dialysis system of the present invention, suitable for use in chronic dialysis or home dialysis;

FIG. 3 shows a schematic of another embodiment of a dialysis system of the present invention, suitable for use in home dialysis or use as a portable dialysis system;

FIG. 4 shows a schematic of an auxiliary module for a dialysis system which can be used to control blood coagulation during dialysis;

FIGS. 5a and 5b show schematics of other embodiments of a dialysis system of the present invention, suitable for use in peritoneal dialysis.

### DETAILED DESCRIPTION

Preferred embodiments of the present invention will now be described in more detail with reference to the accompanying drawings.

Peristaltic pumps of linear build with piezoelectric actuators (hereinafter abbreviated as “piezo pumps”) which are suitable for use in the dialysis systems of the present invention are known in the art in principle. For instance, pumps as disclosed in GB-A 2 238 833 or WO 97/42412, respectively, may be used. It is also conceivable, although less preferred, to use pumps of the type disclosed in WO 2004/071684 A1, U.S. Pat. No. 4,432,699 A or DE 10 2007 019 433 A1, respectively.

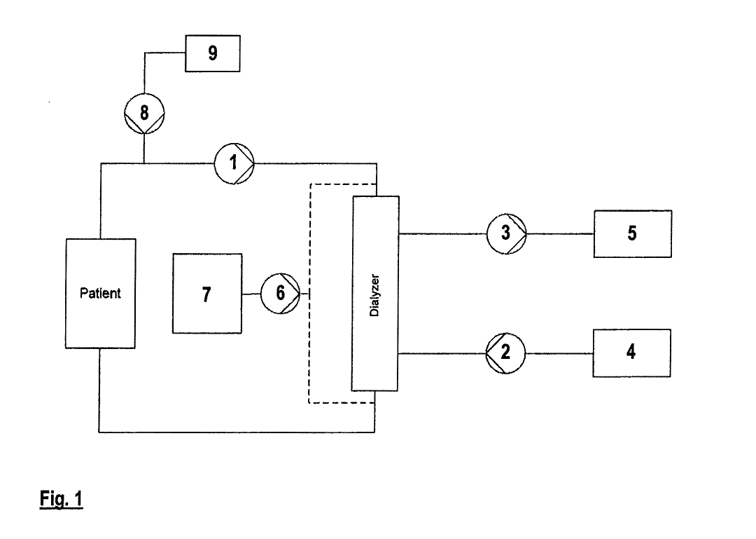

FIG. 1 shows a schematic of an embodiment of a dialysis system of the present invention, suitable for use in intense care. A piezo pump (1) is provided for pumping the patient's blood through the extracorporeal cicuit. Piezo pumps (2,3) are provided for pumping dialysate from a source of dialysate (4), e.g., a bag or a device for on-line preparation of dialysate, through the dialyzer and into a container (5) for spent dialysate. A further piezo pump (6) is provided for feeding substitution fluid from a source (7), e.g., a reservoir or a device for on-line preparation of substitution fluid, to the extracorporal blood circuit, if required, e.g., when the dialysis system is operated in hemodiafiltration modus (HDF) or hemofiltration modus (HF), optionally upstream of the dialyzer (“pre-dilution mode”) or downstream of the dialyzer (“post-dilution mode”). A further piezo pump (8) is provided for feeding infusion solutions (for instance, drug solutions) from a source (9), e.g., a reservoir, to the extracorporeal blood circuit.

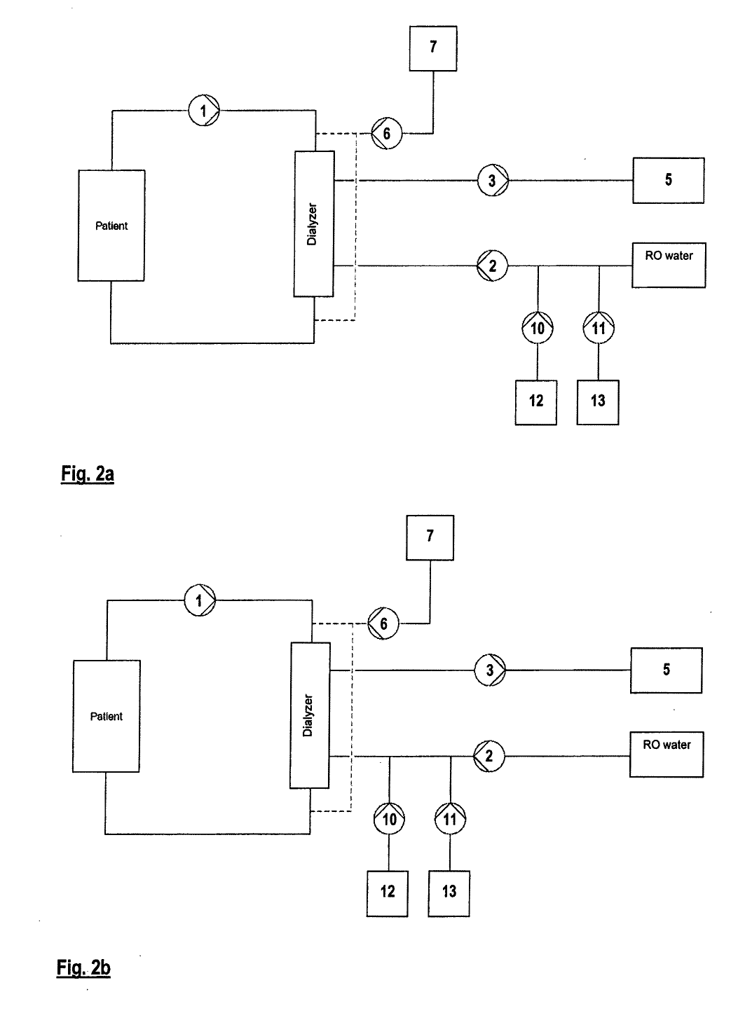

FIGS. 2a and 2b show schematics of embodiments of a dialysis system of the present invention, suitable for use in chronic dialysis or home dialysis. A piezo pump (1) is provided for pumping the patient's blood through the extracorporeal cicuit. Piezo pumps (2,3) are provided for pumping dialysate through the dialyzer and into a container (5) for spent dialysate. In this embodiment, the dialysate is prepared from water demineralized by reverse osmosis (RO water) and dialysis concentrates (12,13) which are admixed via piezo pumps (10,11). The concentrates can be admixed upstream of the first dialysate pump (2), as shown in FIG. 2a, or downstream, as shown in FIG. 2b. A further piezo pump (6) is provided for feeding substitution fluid from a source (7 to the extracorporal blood circuit, if required, e.g., when the dialysis system is operated in hemodiafiltration modus (HDF) or hemofiltration modus (HF), optionally upstream of the dialyzer (“pre-dilution mode”) or downstream of the dialyzer (“post-dilution mode”). Alternatively, piezo pump (6) can also be used for feeding an infusion solution (for instance, drug solutions) instead of substitution fluid from source (7) to the extracorporeal blood circuit.

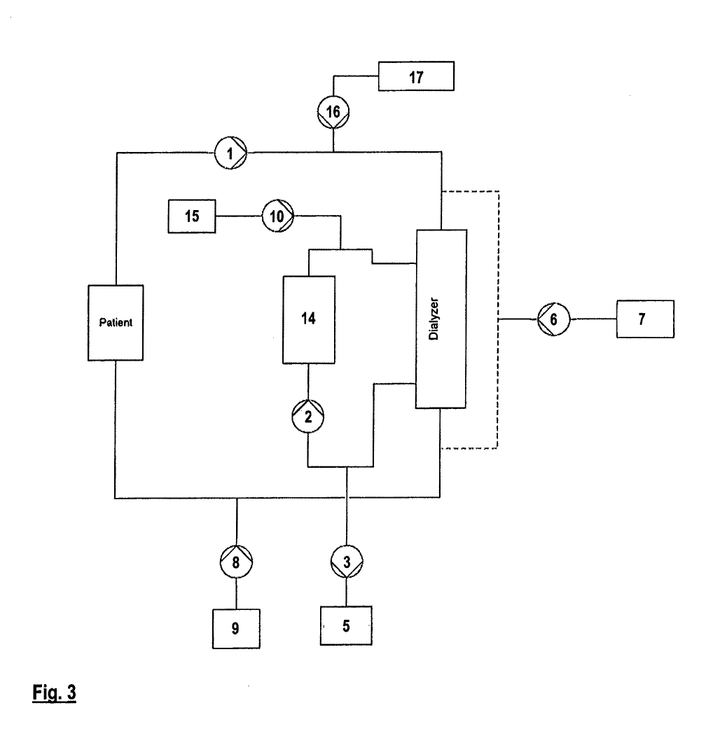

FIG. 3 shows a schematic of another embodiment of a dialysis system of the present invention, suitable for use in home dialysis or use as a portable dialysis system. Because of their small installation size, their low weight and low power consumption, piezo pumps are particularly suitable for the construction of portable dialysis systems, as they allow for a compact and low weight design of the device, and electrical power supply via accumulators or batteries is possible without the total weight of the complete system becoming too high. A piezo pump (1) is provided for pumping the patient's blood through the extracorporeal cicuit.

Piezo pump (2) is provided for pumping dialysate through the dialysate circuit, which in this embodiment also comprises means for dialysate regeneration (14) in addition to the dialyzer. Piezo pump (3) is provided for pumping spent dialysate discharged from the dialysate circuit into a container (5) for spent dialysate. In this embodiment, electrolyte solution from a source (15), e.g., a reservoir, is added to the dialysate via piezo pump (10) to adjust the electrolyte content of the dialysate. A further piezo pump (6) is provided for feeding substitution fluid from a source (7), e.g., a reservoir, to the extracorporal blood circuit, if required, e.g., when the dialysis system is operated in hemodiafiltration modus (HDF) or hemofiltration modus (HF), optionally upstream of the dialyzer (“pre-dilution mode”) or downstream of the dialyzer (“post-dilution mode”). A further piezo pump (8) is provided for feeding infusion solutions (for instance, drug solutions) from a source (9) to the extracorporeal blood circuit. A further piezo pump (16) is provided for adding coagulation-inhibiting agents like heparin from a corresponding source (17) to the blood of the patient in the extracorporeal blood circuit before it enters the the dialyzer.

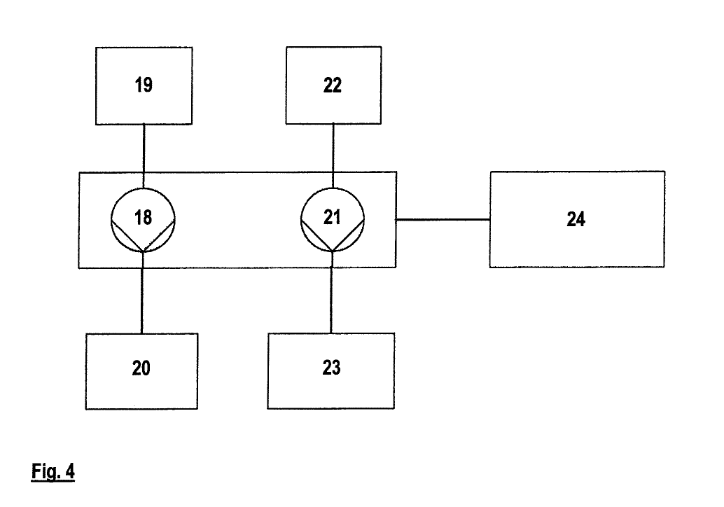

FIG. 4 shows a schematic of an auxiliary module for a dialysis system which can be used to control blood coagulation during dialysis. A piezo pump (18) is provided for pumping citrate solution from a source (19), e.g., a reservoir, into an arterial bloodline (20), in order to lower the coagulation tendency of the patient's blood before it enters the dialyzer. A further piezo pump (21) is provided for pumping a solution comprising calcium ions from a source (22), e.g., a reservoir, into a venous bloodline (20), in order to increase the coagulation tendency of the patient's blood again after it leaves the dialyzer. Means (24) for controlling the piezo pumps (18,21) are provided for controlling the function of the pumps. These means can, e.g., be an interface which connects the auxiliary module with the dialysis monitor. This allows for synchronization of the function of the piezo pumps (18,21) among each other as well as with the flow rates in the extracorporeal blood circuit. The connection between the auxiliary module and the dialysis monitor can be effected, e.g., by leads or wireless, e.g., via W-LAN.

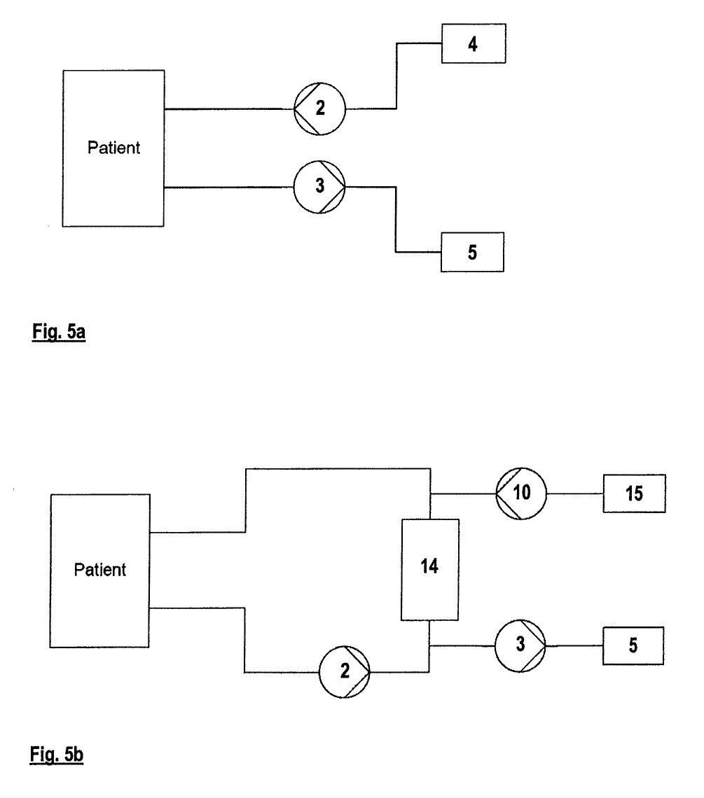

FIGS. 5a and 5b show schematics of other embodiments of a dialysis system of the present invention, suitable for use in peritoneal dialysis. FIG. 5a shows an embodiment, in which piezo pump (2) is provided for pumping dialysate from a dialysate source (4), e.g., a bag, into the peritoneum of the patient, and piezo pump (3) is provided for pumping the dialysate leaving the peritoneum of the patient into a container (5) for spent dialysate. FIG. 5b shows an embodiment, in which piezo pump (2) is provided for pumping dialysate through the dialysate circuit, which in this embodiment comprises means for dialysate regeneration (14). Piezo pump (3) is provided for pumping spent dialysate discharged from the dialysate circuit into a container (5) for spent dialysate. In this embodiment, a solution containing electrolyte and glucose from a source (15), e.g., a reservoir, is added to the dialysate after regeneration via piezo pump (10) to adjust the electrolyte and glucose content of the dialysate.

1. A dialysis system comprising at least one peristaltic pump of linear build with piezoelectric actuators.

2. The dialysis system of claim 1, comprising at least one peristaltic pump of linear build with piezoelectric actuators, said pump being disposed for pumping blood.

3. The dialysis system of claim 1, comprising at least one peristaltic pump of linear build with piezoelectric actuators, said at least one pump being disposed for pumping dialysate.

4. The dialysis system of claim 1, comprising at least one peristaltic pump of linear build with piezoelectric actuators, said pump being disposed for pumping substitution fluid.

5. The dialysis system of claim 1, comprising at least one peristaltic pump of linear build with piezoelectric actuators, said pump being disposed for pumping infusion fluid.

6. The dialysis system of claim 1, comprising at least one peristaltic pump of linear build with piezoelectric actuators, said at least one pump being disposed for pumping dialysis concentrate.

7. A dialysis system comprising a dialyzer connected to an extracorporeal blood circuit and a dialysate circuit, wherein a peristaltic pump of linear build with piezoelectric actuators for pumping blood is provided in said extracorporeal blood circuit; wherein said extracorporeal blood circuit optionally is connected to a source of substitution fluid by a peristaltic pump of linear build with piezoelectric actuators; wherein at least one peristaltic pump of linear build with piezoelectric actuators for pumping dialysate is provided in said dialysate circuit.

8. A device for peritoneal dialysis, comprising a dialysate circuit comprising at least one peristaltic pump of linear build with piezoelectric actuators for pumping dialysate; and means for dialysate regeneration optionally being provided in said dialysate circuit.

9. A device for controlling blood coagulation, comprising a source for citrate solution connected to a peristaltic pump of linear build with piezoelectric actuators, said pump being connected to an arterial bloodline; a source for a solution comprising calcium ions connected to a peristaltic pump of linear build with piezoelectric actuators, said pump being connected to a venous bloodline; and means for controlling said peristaltic pumps.

10. A dialysis system comprising a dialyzer connected to an extracorporeal blood circuit and a dialysate circuit, wherein a peristaltic pump of linear build with piezoelectric actuators for pumping blood is provided in said extracorporeal blood circuit; wherein said extracorporeal blood circuit is connected to a source of infusion fluid by a peristaltic pump of linear build with piezoelectric actuators; wherein at least one peristaltic pump of linear build with piezoelectric actuators for pumping dialysate is provided in said dialysate circuit.

11. A dialysis system comprising a dialyzer connected to an extracorporeal blood circuit and a dialysate circuit, wherein a peristaltic pump of linear build with piezoelectric actuators for pumping blood is provided in said extracorporeal blood circuit; wherein said extracorporeal blood circuit is connected to a source of coagulation inhibitors by a peristaltic pump of linear build with piezoelectric actuators; wherein at least one peristaltic pump of linear build with piezoelectric actuators for pumping dialysate is provided in said dialysate circuit.

12. A dialysis system comprising a dialyzer connected to an extracorporeal blood circuit and a dialysate circuit, wherein a peristaltic pump of linear build with piezoelectric actuators for pumping blood is provided in said extracorporeal blood circuit; wherein at least one peristaltic pump of linear build with piezoelectric actuators for pumping dialysate is provided in said dialysate circuit; wherein said dialysate circuit is connected to a source of dialysis concentrate or electrolyte solution by a peristaltic pump of linear build with piezoelectric actuators.

13. A dialysis system comprising a dialyzer connected to an extracorporeal blood circuit and a dialysate circuit, wherein a peristaltic pump of linear build with piezoelectric actuators for pumping blood is provided in said extracorporeal blood circuit; wherein at least one peristaltic pump of linear build with piezoelectric actuators for pumping dialysate is provided in said dialysate circuit; wherein means for dialysate regeneration are provided in said dialysate circuit.

14. A device for peritoneal dialysis according to claim 8, said dialysate circuit being connected to a source of electrolyte and glucose solution by a peristaltic pump of linear build with piezoelectric actuators.
